(19) United States
(12) Patent Application Publication
(10) Pub. No.: US 2016/0068454 A1
(43) Pub. Date: Mar. 10, 2016

(54) REACTOR AND PROCESS FOR PARAFFIN DEHYDROGENATION TO OLEFINS

(71) Applicant: SAUDI BASIC INDUSTRIES CORPORATION, Riyadh (SA)

(72) Inventors: Zeeshan Nawaz, Riyadh (SA); Faisal Baksh, Riyadh (SA)

(21) Appl. No.: 14/783,005

(22) PCT Filed: Apr. 4, 2014
(86) PCT No.: PCT/IB2014/060444

§ 371 Date: Oct. 8, 2015

Related U.S. Application Data
(60) Provisional application No. 61/809,471, filed on Apr. 8, 2013.

(51) Int. Cl. C07C 5/333; B01J 8/24; B01J 8/18; B01J 8/26

(52) U.S. Cl. CPC C07C 5/3337; C07C 5/3335; B01J 8/26; B01J 8/24; B01J 8/1836; C07C 2523/26; C07C 2523/42; B01J 2208/00168

(57) ABSTRACT

Disclosed herein is a method and apparatus for dehydrogenation of a paraffin comprising: providing a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space; activating a deactivated catalyst present in the regenerator reactor by performing a exothermic catalyst regeneration reaction to produce an activated catalyst and heat; transferring the heat to the integrated fluidized bed reactor; and dehydrogenating a paraffm present in the integrated fluidized bed reactor by performing an endothermic reaction with a catalyst, the paraffm, and at least a portion of the transferred heat to forma dehydrogenation product.

### BACKGROUND

Paraffin dehydrogenation processes can generally be classified as either oxidative or non-oxidative. Oxidative dehydrogenation processes can suffer from, inter alia, the disadvantages of high exothermicity and low desired product selectivity at high conversion. Non-oxidative processes, including direct dehydrogenation, suffer from needing a continuous heat supply (due to the endothermic reaction) and frequent catalyst regeneration. However, the direct dehydrogenation is useful for the production of high demand products, such as propylene or iso-butene. Moreover, the effective reactor performance and process reliability largely depends upon the heat requirement for endothermic reaction.

Although fluidized bed (FBD) reactors have been commercialized, all known reactors have either a separate reactor for catalyst regeneration that is located outside of the reactor or require a third zone. Accordingly, what is needed is a fluidized bed reactor and process that do not suffer the drawbacks of the previous FBD reactions and processes, for example, for endothermic reactions.

### SUMMARY

In accordance with the purpose of the invention, as embodied and broadly described herein, the invention, in one aspect, relates to a method for dehydrogenation of a paraffin comprising providing a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space; activating a deactivated catalyst present in the regenerator reactor by performing a exothermic catalyst regeneration reaction to produce an activated catalyst and heat; transferring the heat to the integrated fluidized bed reactor; and dehydrogenating a paraffin present in the integrated fluidized bed reactor by performing an endothermic reaction with a catalyst, the paraffin, and at least a portion of the transferred heat to forma dehydrogenation product.

In accordance with the purpose of the invention, as embodied and broadly described herein, the invention, in one aspect, relates to an apparatus for dehydrogenation of a paraffin comprising a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 is a schematic drawing of an apparatus of the present invention.

FIG. 2 is a schematic drawing of a apparatus of the present invention.

FIG. 3 is a schematic view of a process flow scheme of the present invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention.

Before the present apparatus and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a paraffin” includes mixtures of two or more paraffins.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. Dehydrogenation of Paraffin

Methods for dehydrogenation of paraffins are provided. In one aspect, a method is disclosed for dehydrogenation of a paraffin comprising: providing a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space; activating a deactivated catalyst present in the regenerator reactor by performing a exothermic catalyst regeneration reaction to produce an activated catalyst and heat; transferring the heat to the integrated fluidized bed reactor; and dehydrogenating a paraffin present in the integrated fluidized bed reactor by performing an endothermic reaction with a catalyst, the paraffin, and at least a portion of the transferred heat to forma dehydrogenation product.

In one aspect, the method further comprises transferring a deactivated catalyst from the integrated fluidized bed reactor to the regenerator reactor. The transfer of the deactivated catalyst from the integrated fluidized bed reactor to the regenerator reactor can be done by transferring the deactivated catalyst through a striping zone that is in communication, such as fluid communication, with the integrated fluidized bed reactor, and wherein the striping zone is connected to the regenerator reactor via a first connector. The first connector can comprise a gas injection system, which provides a stream of gas (air and or oxygen) that facilitates the transfer of the deactivated catalyst from the striping zone to the regenerator reactor and facilitates the regeneration (reactivation of the deactivated catalyst).

A deactivated catalyst is a catalyst that has lost at least some of its activity towards its ability to dehydrogenate a paraffin. The loss in activity can be due to coke buildup on the catalyst and/or support of the catalyst, which can occur during the dehydrogenation of a paraffin. A deactivated catalyst can be reactivated in the regenerator reactor, thereby producing an activated catalyst.

The activation of the deactivated catalyst is performed by a exothermic catalyst regeneration reaction. Accordingly, heat is generated by the exothermic catalyst regeneration reaction. The heat or at least a portion of the heat is transferred from the regenerator reactor, where the exothermic catalyst regeneration reaction occurs, to the integrated fluidized bed reactor. The walls of the regenerator reactor can be made of a material that allows for heat transfer, such as a metal material. Thus, the transfer of heat from the regenerator reactor can be done by transferring the heat through the walls of the regenerator reactor to the integrated fluidized bed reactor.

The dehydrogenation of a paraffin in the integrated fluidized bed reactor can be performed by an endothermic reaction with a catalyst, the paraffin, and at least a portion of the transferred heat to form a dehydrogenation product. Accordingly, at least a portion of the heat produced by the exothermic catalyst regeneration reaction is used to assist and drive the endothermic reaction to dehydrogenate a paraffin in the integrated fluidized bed reactor. As such, energy is recycled in the reactor and the need for external or internal heating sources in the reactor are reduced. The paraffin is dehydrogenated at a temperature ranging from 490° C. to 640° C. At least a portion of the energy (heat) required to reach and maintain such temperature in the integrated fluidized bed reactor comes from the energy (heat) generated by the exothermic catalyst regeneration reaction in the regenerator reactor.

In one aspect, the method further comprises transferring the activated catalyst from the regenerator reactor to the integrated fluidized bed reactor. Transferring the activated catalyst to the integrated fluidized bed reactor can be performed via a catalyst collection zone, wherein the regenerator reactor has an outlet that is positioned in the catalyst collection zone, and wherein the catalyst collection zone is connected to the integrated fluidized bed reactor via a second connector. Accordingly, the reactor is capable of activating and recycling deactivated catalyst within the reactor. Such recycle process reduces the cost since less new catalyst is needed and also provides for a method that can be performed continuously without the need for introducing new catalyst and/or replacing used deactivated catalyst.

In one aspect, the method further comprises introducing paraffin into the integrated fluidized bed reactor. The method can comprise continuously introducing paraffin into the integrated fluidized bed reactor.

In one aspect, the method can further comprise removing the dehydrogenation product from the dehydrogenation reactor. For example, the dehydrogenation product can be removed from the integrated fluidized bed reactor.

In one aspect, paraffin comprises from three to six carbons. In another aspect, the paraffin comprises an alkyl chain, such as a alkyl chain having three to six carbons. In another aspect, the paraffin comprises propane, n-butane, isobutane, n-pentane, 2-methylbutane, n-hexane, 2-methylpentane, 3-methylpentane, 2,3-dimethylbutane, or 2,2,-dimethylbutane, or a combination thereof.

In another aspect, the dehydrogenation product comprises an olefin, such as a mono-olefin. Examples include, but are not limited to, propene, n-butene, n-pentene, iso-pentene, n-hexene, or iso-hexene, or a combination thereof. In another aspect, the dehydration product comprises but-1-ene, cis-2-butene, trans-2-butene, pent-1-ene, pent-2-ene, 2-methylbut-1-ene, 3-methylbut-1-ene, 2-methyl-but-2-ene, hex-1-ene, cis-hex-2-ene, trans-hex-2-ene, cis-hex-3-ene, trans-hex-3-ene, 2-methylpent-1-ene, 3-methylpent-1-ene, 4-methylpent-1-ene, cis-2-methylpent-2-ene, trans-2-methylpent-2-ene, cis-3-methylpent-3-ene, trans-3-methylpent-2-ene, cis-3-methylpent-2-ene, trans-3-methylpent-2-ene, cis-4-methylpent-2-ene, trans-4-methylpent-2-ene, 2,3-dimethylbut-1-ene, 3,3-dimethylbut-1-ene, 2,3-dimethylbut-2-ene, or 2-ethylbut-1-ene, or a combination thereof.

In one aspect, the paraffin is dehydrogenated at a temperature ranging from 490° C. to 655° C., including exemplary values of 495° C., 500° C., 505° C., 510° C., 515° C., 520° C., 525° C., 530° C., 535° C., 540° C., 545° C., 550° C., 555° C., 560° C., 565° C., 570° C., 575° C., 580° C., 585° C., 590° C., 595° C., 600° C., 605° C., 610° C., 615° C., 620° C., 625° C., 630° C., 635° C., 640° C., 645° C., and 650° C. In still further aspects, the temperature can be in a range derived from any two of the above listed exemplary temperatures. For example, the temperature can range from 530° C. to 640° C. or from 540° C. to 630° C.

In another aspect, the paraffin is dehydrogenated at a pressure ranging from 0.1atmospheres (atm) (0.01 MegaPascals (MPa)) to 3 atmospheres (0.3 MPa), including exemplary values of 0.2 atm (0.02 MPa), 0.3 atm (0.03 MPa), 0.4 atm (0.04 MPa), 0.5 atm (0.05 MPa), 0.6 atm (0.06 MPa), 0.7 atm (0.07 MPa), 0.8 atm (0.08 MPa), 0.9 atm (0.09 MPa), 1 atm (0.1 MPa), 1.2 atm (0.12 MPa), 1.4 atm (0.14 MPa), 1.6 atm (0.16 MPa), 1.8 atm (0.18 MPa), 2 atm (0.2 MPa), 2.2 atm (0.22 MPa), 2.4 atm, 2.6 atm (0.26 MPa), and 2.8 atm (0.28 MPa). In still further aspects, the pressure can be in a range derived from any of the two above listed exemplary pressures. For example, the pressure can range from 0.2 atm (0.02 MPa) to 2.8 atm (0.28 MPa) or from 0.5 atm (0.05 MPa) to 2.5 atm (0.25 MPa).

In one aspect, the paraffin dehydrogenation is measured using GHSV. The GHSV will further adjust to the desired hold up in the inventive system. As used herein, GHSV refers to the gas hourly space velocity and allows for relating the gas flow rate to the reactor volume. GHSV indicates how many reactor volumes of feed can be treated in a unit time, and it is typically regarded as the reciprocal of the reactor space time. These operation parameters vary from feed to feed (or feed composition), catalyst to catalyst (or active metal loading content), mode of operation, and size of design (desired hydrodynamics).

In another aspect, the methods can comprise a GHSV pressure ranging from 0.5-2 atm (0.05-0.2 MPa), including exemplary values of 0.75 atm (0.075 MPa), 1 atm (0.1 MPa), 1.25 atm (0.125 MPa), 1.5 atm (0.15 MPa), and 1.75 (0.175 MPa) atm. In still further aspects, the GHSV pressure can be in a range derived from any two of the above listed exemplary GHSV values. For example, the GHSV pressure can range from 0.75 atm (0.075 MPa) to 1.25 atm (0.125 MPa). In a further aspect, the GHSV pressure can be 1 atm (0.1 MPa).

In still another aspect, the methods disclosed herein can comprise a paraffin being dehydrogenated over a Cr-based catalyst at a GHSV ranging from 100 h⁻¹ to 10000 h⁻¹, including exemplary values of 200 h⁻¹, 300 h⁻¹, 400 h⁻¹, 500 h⁻¹, 600 h⁻¹, 700 h⁻¹, 800 h⁻¹, 900 h⁻¹, 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 3000 h⁻¹, 4000 h⁻¹, 5000 h⁻¹, 6000 h⁻¹, 7000 h⁻¹, 8000 h⁻¹, and 9000 h⁻¹. In still further aspects, the GHSV can be in a range derived from any two of the above listed exemplary GHSV values. For example, the GHSV can be 200 h⁻¹ to 9000 h⁻¹ or from 500 h⁻¹ to 5000 h⁻¹. In one aspect, the paraffin is dehydrogenated over Cr₂O₃/Al₂O₃. In a further aspect, the Cr loading in the methods can range from range of 8 wt % to 22 wt %.

In another aspect, the methods disclosed herein can comprise a paraffin being dehydrogenated over a Pt-based catalyst at a GHSV ranging from 1 h⁻¹ to 100 h⁻¹, including exemplary values of 2 h⁻¹, 3 h⁻¹, 4 h⁻¹, 5 h⁻¹, 6 h⁻¹, 7 h⁻¹, 8 h⁻¹, 9 h⁻¹, 10 h⁻¹, 11 h⁻¹, 12 h⁻¹, 13 h⁻¹, 14 h⁻¹, 15 h⁻¹, 16 h⁻¹, 17 h⁻¹, 18 h⁻¹, 19 h⁻¹, 20 h⁻¹, 30 h⁻¹, 40 h⁻¹, 50 h⁻¹, 60 h⁻¹, 70 h⁻¹, 80 h⁻¹, and 90 h⁻¹. In still further aspects, the GHSV can be in a range derived from any two of the above listed exemplary GHSV values. For example, the GHSV range from 2 h⁻¹ to 90 h⁻¹, from 1 h⁻¹ to 20 h⁻¹, or from 5 h⁻¹ to 50 h⁻¹ for a Pt-based catalyst.

As used herein, residence time refers to the average amount of time that the reacting catalyst spends in the dehydrogenation reactor. The residence time of the catalyst can also be called the catalyst circulation rate.

In one aspect, the catalyst circulation rate (residence time of the catalyst) in the integrated fluidized bed reactor, for the Cr-based catalyst ranges from 2 minutes to 22 minutes, including exemplary values of 5 minutes, 7 minutes, 10 minutes, 13 minutes, 15 minutes, 17 minutes, and 20 minutes. In still further aspects, the catalyst circulation rate can be in a range derived from any two of the above listed exemplary catalyst circulation rate values. For example, the catalyst circulation rate can range from 5 minutes to 20 minutes.

In one aspect, the catalyst circulation rate (residence time of the catalyst) in the integrated fluidized bed reactor, for the Pt-based catalyst ranges from 1 hour to 8 hours (hr), including exemplary values of 1.5 hr, 2 hr, 2.5 hr, 3 hr, 3.5 hr, 4 hr, 4.5 hr, 5 hr, 5.5 hr, 6 hr, 6.5 hr, 7 hr, and 7.5 hr. In still further aspects, the catalyst circulation rate can be in a range derived from any two of the above listed exemplary catalyst circulation rate values. For example, the catalyst circulation rate can range from 1.5 hours to 6.5 hours or from 2.5 hours to 7.5 hours.

In one aspect, the paraffin is dehydrogenated at a temperature ranging from 490° C. to 640° C., at a pressure ranging from 0.1 atmospheres (0.01 MPa) to 3 atmospheres (0.3 MPa), and a GHSV ranging from 100 h⁻¹ to 10,000 h⁻¹.

In one aspect, the integrated fluidized bed reactor, in the methods disclosed herein, is substantially isothermal. As such, methods by way of the integrated fluidized bed reactor can keep the reactor temperature substantially controlled. The reactor temperature is kept substantially controlled by the system being in contact with an outside thermal reservoir. In another aspect, the outside thermal reservoir is a heat exchanger. In a further aspect, the outside thermal reservoir is a regenerator-riser.

In one aspect, the fresh/recycled paraffin feed comes in contact with the catalyst, such as an active or activated catalyst, in integrated fluidized bed reactor, where the activated catalyst enters from the catalyst collection zone by gravity/drag. In another aspect, the activated catalyst from the regenerator reactor does not need to be transported. In a further aspect, the dehydrogenation occurs on the catalyst surface present in the integrated fluidized bed reactor and the product is separated by cyclones and spent deactivated catalyst is transferred to the regenerator reactor via a connector, which optionally comprises a flow of air or oxygen or steam. Deactivated catalyst can be regenerated by burning coke/heavies depositions at around 600-750° C., then redispersed, if required, and finally reduced either by feed or hydrogen or methane at about 500-650° C. The dehydrogenation of the paraffin in the integrated fluidized bed reactor to form a dehydrogenation product and activating the catalyst in the regenerator reactor can occur simultaneously.

### C. Catalyst

In one aspect, the invention has the ability to use any catalyst appropriate for dehydrogenation. In one aspect, the catalyst comprises a Cr-based catalyst or a Pt-based catalyst, or a combination thereof. In another aspect, the catalyst is present on a support. In a further aspect, the catalyst is modified by a promoter.

In one aspect, the paraffin is dehydrogenated over Cr₂O₃/Al₂O₃.

In a further aspect, the Cr loading can range from range of 8 wt % to 22 wt %, including exemplary values of 9 wt %, 10 wt %, 11 wt %, 14 wt %, 15 wt %, 17 wt %, 18 wt %, 19 wt %, 20 wt %, or 21 wt %. In still further aspects, the Cr loading can be in a range derived from any two of the above listed exemplary Cr loading values. For example, the Cr loading can range from 9 wt % to 21 wt %. In still another aspect, the catalyst is regenerated by burning coke on the catalyst surface at a temperature greater than the average temperature of the reactor in a stream of steam, air, oxygen, and fuel gas. The catalyst regeneration, in the regenerator reactor, is an exothermic process, and can act as a source of heat for the catalyst and to maintain the reactor temperature. The regeneration residence time can depend on the type of catalyst, catalyst loading, and the circulation rate required for the steady operation in the reactor. The fuel gas injection requirement can vary with the severity and the mode of operation. The catalyst regeneration parameters further influence the temperature requirement for catalyst regeneration. As such, the temperature for catalyst regeneration can range from 550° C. to 750° C., including exemplary values of 575° C., 600° C., 625° C., 650° C., 675° C., 700° C., and 725° C. In still further aspects, the temperature for catalyst regeneration can be in a range derived from any two of the above listed exemplary temperatures. For example, the temperature for catalyst regeneration can range from 575° C. to 725° C.

In one aspect, a platinum-based catalyst or a chromium based catalyst can be supported on a non-acid support, such as alumina or silica alumina. In a further aspect, a platinum-based catalyst or a chromium based catalyst can be supported on an acid support, a zeolite, or a metal oxide, or a combination thereof. In another aspect, the dehydration can use a multi-component catalyst and/or a metal oxide. For example, the platinum or the chromium based catalysts on a non-acid support, an acid support, a zeolite, or a metal oxide, or a combination thereof, are recited in U.S. Pat. Nos. 5,132,484; 3,488,402; 2,374,404; and International Publication No. WO/2005/040075, all of which are hereby incorporated in their entirety for the specific purpose of disclosing various platinum or chromium based catalysts on a non-acid support compositions and methods that can be used herein.

In one aspect, the catalyst can be modified by one or more promoters. The promoter can control the stereochemistry of the dehydrogenation reaction. For example, catalysts modified by one or more promoters are recited in U.S. Pat. Nos. 5,198,597; 5,146,034; 3,899,544; 3,679,773; 4,000,210; 4,177,218; 2,814,599 and 3,679,773; Publication Nos. CN200910091226 and PK140812/2010, all of which are hereby incorporated in their entirety for the specific purpose of disclosing various compositions and methods of catalysts modified by one or more promoters that can be used herein.

In another aspect, the catalyst comprises one or more promoters dispersed on aluminum oxide, silicon oxide, or zeolite, or combination thereof. For example, the catalysts comprising one or more promoters dispersed on aluminum oxide, silicon oxide, or zeolite, other metal oxides, or combination thereof are recited in U.S. Pat. Nos. 2,814,599; 3,679,773; 5,416,052; 5,146,034; 3,507,931; 3,551,353; 3,932,554; 4,935,578; and 5,132,479; and CN Pat. No. 1762931, all of which are hereby incorporated in their entirety for the specific purpose of disclosing various compositions and methods of catalysts comprising one or more promoters that can be used herein.

### D. Apparatus

The apparatus disclosed herein can be used in any of the methods disclosed herein. In one aspect, an apparatus for dehydrogenation of paraffins is provided. The apparatus for dehydrogenation of a paraffin comprises a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space.

In one aspect, the apparatus does not comprise an external regenerator reactor. In another aspect, the apparatus does not comprise a zone configured to comprise a gas phase thermal reaction.

The walls of the regenerator reactor can be made of a material that allows for energy (heat) transfer, such as a metal material. Thus, the transfer of heat from the regenerator reactor can be done by transferring the heat through the walls of the regenerator reactor to the integrated fluidized bed reactor.

In one aspect, the dehydrogenation reactor further comprises a striping zone, wherein the striping zone is in communication, such as fluid communication, with the integrated fluidized bed reactor, and wherein the striping zone is connected to the regenerator reactor via a first connector. Accordingly, deactivated catalyst will be transferred from the integrated fluidized bed reactor to the striping zone and then transferred to the regenerator reactor via the first connector.

In one aspect, the first connector comprises a gas injection system. The gas injection system can provide a flow of gas, such as air and/or oxygen through the first connector, thereby pushing the deactivated that has entered to the first connector towards and finally into the regenerator reactor. The gas injection system can comprise a blower. The gas injection system can provide air and/or oxygen that can be used in the regeneration of the deactivated catalyst in the regenerator reactor.

In one aspect, the integrated fluidized bed reactor or the striping zone or a combination thereof comprises an integrated internal baffle or a perforated tray or a combination thereof. For example, the integrated fluidized bed reactor can comprise an integrated internal baffle or a perforated tray or a combination thereof. In another example, the striping zone can comprise an integrated internal baffle or a perforated tray or a combination thereof. The integrated internal baffle and/or perforated tray, present in the integrated fluidized bed reactor or the striping zone, can provide superior holdup and/or well mixed hydrodynamics.

In one aspect, the dehydrogenation reactor further comprises a catalyst collection zone, wherein the regenerator reactor has an outlet that is positioned in the catalyst collection zone, and wherein the catalyst collection zone is connected to the integrated fluidized bed reactor via a second connector. Activated catalyst produced in the regenerator reactor is collected in the catalyst collection zone. The activated catalyst can then be transferred into the integrated fluidized bed reactor via a second connector. The catalyst collection zone can be position above the integrated fluidized bed reactor. Thus, the transfer of the activated catalyst from the catalyst collection zone to the integrated fluidized bed reactor can be a function of gravity.

In one aspect, the first longitudinal axis is parallel to the second longitudinal axis, and optionally, wherein the first longitudinal axis and the second longitudinal axis are axially aligned, thereby forming a common longitudinal axis. For example, the first longitudinal axis and the second longitudinal axis can be axially aligned, thereby forming a common longitudinal axis. Thus, at least a portion of the regenerator reactor can be present in the middle of the space in the integrated fluidized bed reactor.

In one aspect, the integrated fluidized bed reactor comprises a feed distributor or a cleaning ring or a combination thereof. The feed distributor or a cleaning ring or a combination thereof promotes uniform distribution of the paraffin feed into the integrated fluidized bed reactor.

In one aspect, the integrated fluidized bed reactor is configured to contain an endothermic reaction. The endothermic reaction can be the endothermic reaction of dehydrogenating a paraffin present in the integrated fluidized bed reactor by performing an endothermic reaction with a catalyst.

In one aspect, the regenerator reactor is configured to contain an exothermic reaction. The exothermic reaction can be the reaction of activating a deactivated catalyst present in the regenerator reactor by performing a exothermic catalyst regeneration reaction to produce an activated catalyst.

In one aspect, the apparatus does not comprise an external regenerator reactor. Accordingly, the apparatus, in this aspect, does not comprise a regenerator reactor outside of the space in the integrated fluidized bed reactor.

Previous fluidized bed reactors, which should be distinguished from the present invention, have either a separate reactor or catalyst regenerator external to the space in the integrated fluidized bed reactor. Such reactors also involve a process of transferring the catalyst outside of the apparatus for regeneration, or require a third zone. Non-limiting examples of such previous fluidized bed reactors include those disclosed in U.S. Pat. Nos. 7,829,753; 6,362,385; 5,143,886; and 5,633,421 which are incorporated in their entirety for the specific purpose of disclosing fluidized bed reactors with either (1) a separate reactor and catalyst regenerator or (2) a third zone.

In one aspect, the integrated fluidized bed reactor and/or the regenerator reactor act as a riser or as a downcomer.

An integrated fluidized bed reactor with for paraffin dehydrogenation to olefins is disclosed. The integrated fluidized bed reactor design can provide the advantage of sharing regenerator heat to the reactor, conducting endothermic reaction to run reactor substantially isothermal, lower catalyst circulation, and lower attrition. The overall process can enhance paraffin feed conversion and desired product selectivity.

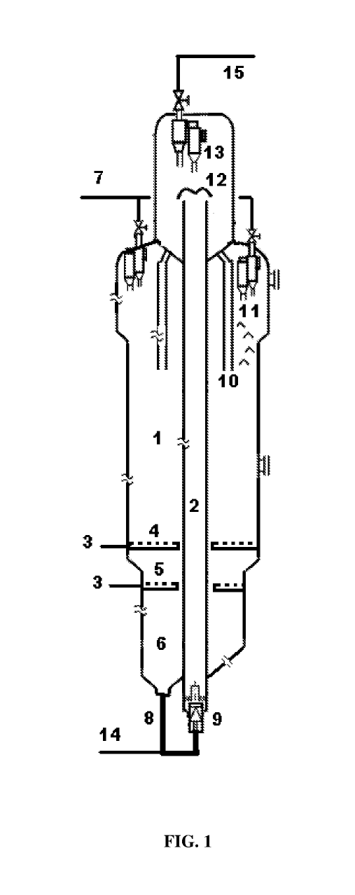

Referring now to FIG. 1, which is one aspect of the apparatus of the invention, the feed comprising one or more alkanes (paraffin) enters through line or a mixture of different paraffin's or some time with other impurities like hydrogen, olefins, etc. 3 to integrated fluidized bed reactor 1, through a feed distributor 4 and/or a cleaning ring(s) 5 (these can further help in distributor efficiency). A line 3 can either be connected to a feed distributor 4 or a cleaning ring(s) 5, as shown in FIG. 1. The reactor comprises both an integrated fluidized bed reactor 1 and a striping zone 6, both equipped with an internal grid to achieve superior gas-solid contact. The dehydrogenation product gas is separated from the catalyst in a series of primary and secondary cyclones 11 products is collected at 7. The reactor also includes a regenerator reactor 2, where deactivated catalyst can be reactivated through an exothermic catalyst regeneration reaction. The catalyst can be regenerated (reactivated) by burning coke/heavies depositions at around 600-750° C., then re-dispersed, if required, and finally reduced either by feed or hydrogen or methane at about 500-650° C. The regenerator reactor 2 is positioned within the space of the integrated fluidized bed reactor 1 so that energy (heat) produced by the exothermic reaction in regenerator reactor 2 can be transferred to the integrated fluidized bed reactor 1. For example, the walls of regenerator reactor 2 can be made from a material that conducts heat, such as a metal material. Deactivated catalyst is transferred from the striping zone 6 via connector 8 to regenerator reactor 2 where the deactivated catalyst is reactivated. The reactivated catalyst is then collected in catalyst collection zone 12, where fresh catalyst can also be added, if needed. The deactivated catalyst is pushed by the air, oxygen, and fuel generated from gas injection system 14, and rushed to the regenerator distributor 9, which distributes the deactivated catalyst into regenerator reactor 2. The reactivated catalyst is then dropped from catalyst collection zone 12 to the integrated fluidized bed reactor 1 through connector 10. The off gasses 15 can be separated from the catalyst by the cyclone separator 13.

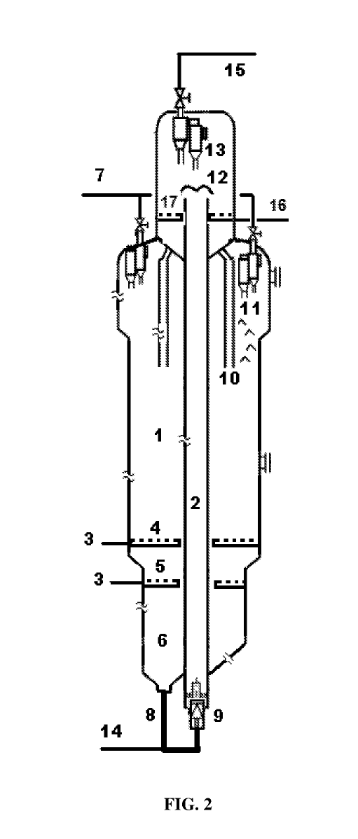

Referring to FIG. 2, which is one aspect of the apparatus of the invention, shows a design of the apparatus, which can depend on the size and capacity of the apparatus. In certain cases if additional reduction of the catalyst is required, the regenerator reactor 2 of FIG. 1 can be equipped with a reducing gas distributor 17 with a gas injection line 16.

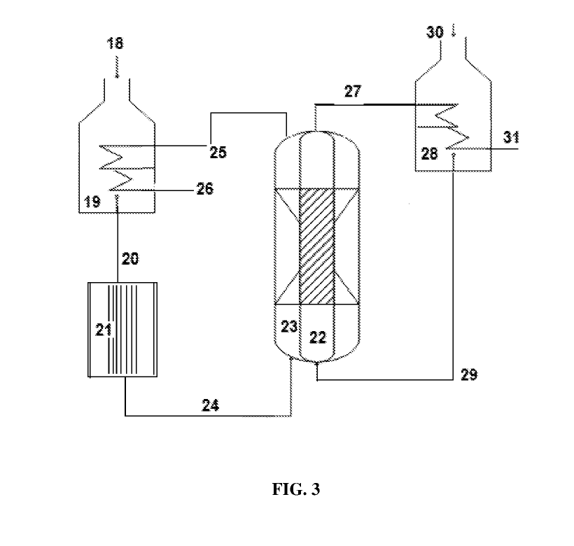

Referring now to FIG. 3, in one aspect, shows an integrated scheme of the overall process of the dehydrogenation of paraffin's using inventive design. The feed of paraffin from line 18 is pre-heated in the gas-gas heat exchanger 19 by exchanging heat from hot product gas 25 coming from reactor. The final cooled product gas 26 is collected after exchanging heat in the gas-gas heat exchanger 19 for further downstream processing. The hot feed from 20 is further heated to desired feed temperature in fired heater 21 if needed, and sent to the integrated fluidized bed reactor 23 by line 24. The final product 26 is collected after exchanging heat in the gas-gas heat exchanger 19. Deactivated catalyst is reactivated in regenerator 22 by hot air from line 29 coming from heat exchanger 28. The air 30 is heated by regenerator effluent stream 27 and evacuated at exit 31.

In one aspect, the paraffin is dehydrogenated at a temperature ranging from 490° C. to 655° C., including exemplary values of 495° C., 500° C., 505° C., 510° C., 515° C., 520° C., 525° C., 530° C., 535° C., 540° C., 545° C., 550° C., 555° C., 560° C., 565° C., 570° C., 575° C., 580° C., 585° C., 590° C., 595° C., 600° C., 605° C., 610° C., 615° C., 620° C., 625° C., 630° C., 635° C., 640° C., 645° C., and 650° C. In still further aspects, the temperature can be in a range derived from any two of the above listed exemplary temperatures. For example, the temperature can range from 530° C. to 640° C. or from 540° C. to 630° C.

In another aspect, the paraffin is dehydrogenated at a pressure ranging from 0.1 atmospheres to 3 atmospheres (0.01 MPa to 0.3 MPa), including exemplary values of 0.2 atm (0.02 MPa), 0.3 atm (0.03 MPa), 0.4 atm (0.04 MPa), 0.5 atm (0.05 MPa), 0.6 atm (0.06 MPa), 0.7 atm (0.07 MPa), 0.8 atm (0.08 MPa), 0.9 atm (0.09 MPa), 1 atm (0.1 MPa), 1.2 atm (0.012 MPa), 1.4 atm (0.014 MPa), 1.6 atm (0.016 MPa), 1.8 atm (0.018 MPa), 2 atm (0.2 MPa), 2.2 atm (0.22 MPa), 2.4 atm (0.24 MPa), 2.6 atm (0.26 MPa), and 2.8 atm (0.28 MPa). In still further aspects, the pressure can be in a range derived from any of the two above listed exemplary pressures. For example, the pressure can range from 0.2 atm (0.02 MPa) to 2.8 atm (0.028 MPa) or from 0.5 atm (0.05 MPa) to 2.5 atm (0.025 MPa).

In one aspect, the paraffin dehydrogenation is measured using GHSV. The GHSV will further adjust to the desired hold up in the inventive system. As used herein, GHSV refers to the gas hourly space velocity and allows for relating the gas flow rate to the reactor volume. GHSV indicates how many reactor volumes of feed can be treated in a unit time, and it is commonly regarded as the reciprocal of the reactor space time. These operation parameters vary from feed to feed (or feed composition), catalyst to catalyst (or active metal loading content), mode of operation, and size of design (desired hydrodynamics).

In another aspect, the GHSV pressure ranges from 0.5-2 atm (0.05-0.2 MPa), including exemplary values of 0.75 atm (0.075 MPa), 1 atm (0.1 MPa), 1.25 atm (0.0125 MPa), 1.5 atm (0.015 MPa), and 1.75 atm (0.0175 MPa). In still further aspects, the GHSV pressure can be in a range derived from any two of the above listed exemplary GHSV values. For example, the GHSV pressure can range from 0.75 atm to 1.25 atm (0.075 MPa to 0.125 MPa). In a further aspect, the GHSV pressure can be 1 atm (0.01 MPa).

In still another aspect, the paraffin is dehydrogenated over a Cr-based catalyst at a GHSV ranging from 100 h⁻¹ to 10000 h⁻¹, including exemplary values of 200 h⁻¹, 300 h⁻¹, 400 h⁻¹, 500 h⁻¹, 600 h⁻¹, 700 h⁻¹, 800 h⁻¹, 900 h⁻¹, 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 3000 h⁻¹, 4000 h⁻¹, 5000 h⁻¹, 6000 h⁻¹, 7000 h⁻¹, 8000 h⁻¹, and 9000 h⁻¹. In still further aspects, the GHSV can be in a range derived from any two of the above listed exemplary GHSV values. For example, the GHSV can be 200 h⁻¹ to 9000 h⁻¹ or from 500 h⁻¹ to 5000 h⁻¹. In one aspect, the paraffin is dehydrogenated over Cr₂O₃/Al₂O₃. In a further aspect, the Cr loading can range from range of 8 wt % to 22 wt %.

In another aspect, the paraffin is dehydrogenated over a Pt-based catalyst at a GHSV ranging from 1 h⁻¹ to 100 h⁻¹, including exemplary values of 2 h⁻¹, 3 h⁻¹, 4 h⁻¹, 5 h⁻¹, 6 h⁻¹, 7 h⁻¹, 8 h⁻¹, 9 h⁻¹, 10 h⁻¹, 11 h⁻¹, 12 h⁻¹, 13 h⁻¹, 14 h⁻¹, 15 h⁻¹, 16 h⁻¹, 17 h⁻¹, 18 h⁻¹, 19 h⁻¹, 20 h⁻¹, 30 h⁻¹, 40 h⁻¹, 50 h⁻¹, 60 h⁻¹, 70 h⁻¹, 80 h⁻¹, and 90 h⁻¹. In still further aspects, the GHSV can be in a range derived from any two of the above listed exemplary GHSV values. For example, the GHSV range from 2 h⁻¹ to 90 h⁻¹, from 1 h⁻¹ to 20 h⁻¹, or from 5 h⁻¹ to 50 h⁻¹ for a Pt-based catalyst.

As used herein, residence time refers to the average amount of time that the reacting catalyst spends in the dehydrogenation reactor. The residence time of the catalyst can also be called the catalyst circulation rate.

In one aspect, the catalyst circulation rate (residence time of the catalyst) in the regenerator reactor, for the Cr-based catalyst ranges from 2 minutes to 22 minutes, including exemplary values of 5 minutes, 7 minutes, 10 minutes, 13 minutes, 15 minutes, 17 minutes, and 20 minutes. In still further aspects, the catalyst circulation rate can be in a range derived from any two of the above listed exemplary catalyst circulation rate values. For example, the catalyst circulation rate can range from 5 minutes to 20 minutes.

In one aspect, the catalyst circulation rate (residence time of the catalyst) in the integrated fluidized bed reactor, for the Pt-based catalyst ranges from 1 hour to 8 hours, including exemplary values of 1.5 hr, 2 hr, 2.5 hr, 3 hr, 3.5 hr, 4 hr, 4.5 hr, 5 hr, 5.5 hr, 6 hr, 6.5 hr, 7 hr, and 7.5 hr. In still further aspects, the catalyst circulation rate can be in a range derived from any two of the above listed exemplary catalyst circulation rate values. For example, the catalyst circulation rate can range from 1.5 hours to 6.5 hours or from 2.5 hours to 7.5 hours.

In one aspect, the paraffin is dehydrogenated at a temperature ranging from 490° C. to 640° C., at a pressure ranging from 0.1 atmospheres to 3 atmospheres, and a GHSV ranging from 100 h⁻¹ to 10,000 h⁻¹.

In one aspect, the integrated fluidized bed reactor comprises an endothermic reaction. In another aspect, the regenerator reactor comprises an exothermic reaction. One uniqueness of the proposed design is to benefit from exothermic reaction in a regenerator for an endothermic reaction. Moreover these transfers consume less energy.

In one aspect, the fluidized bed is substantially isothermal. As such, the fluidized bed can keep the reactor temperature substantially controlled. The reactor temperature is kept substantially controlled by the system being in contact with an outside thermal reservoir. In another aspect, the outside thermal reservoir is a heat exchanger. In a further aspect, the outside thermal reservoir is a regenerator-riser.

In one aspect, the integrated fluidized bed reactor comprises an endothermic dehydrogenation reaction and requires temperature from 500-640° C. depending on the feed composition. On the other hand the regeneration temperature is relatively higher as it is a coke combustion reaction. In another aspect, the regenerator reactor comprises an exothermic reaction. In one aspect, the integrated fluidized bed reactor comprises an endothermic reaction, while the regenerator reactor comprises an exothermic reaction.

In one aspect, the fresh/recycled paraffin feed comes in contact with fluidizable active catalyst in the integrated fluidized bed reactor, where the regenerated catalyst enters from the top by gravity/drag from the catalyst collection zone. In another aspect, the regenerated catalyst from the regenerator reactor does not need to be transported by mechanical means to the integrated fluidized bed reactor. In a further aspect, the dehydrogenation occurs on the catalyst surface in integrated fluidized bed reactor and the product is separated by cyclones and deactivated catalyst rushed to the regenerator reactor by regeneration sources of air or oxygen or steam from the gas injection system. Deactivated catalyst can be regenerated by burning coke/heavies depositions at around 600-750° C., then redispersed, if required, and finally reduced either by feed or hydrogen or methane at about 500-650° C.

### E. Aspects

The disclosed compositions and methods include at least the following aspects.

Aspect 1: A method for dehydrogenation of a paraffin comprising: providing a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space; activating a deactivated catalyst present in the regenerator reactor by performing a exothermic catalyst regeneration reaction to produce an activated catalyst and heat; transferring the heat to the integrated fluidized bed reactor; and dehydrogenating a paraffin present in the integrated fluidized bed reactor by performing an endothermic reaction with a catalyst, the paraffin, and at least a portion of the transferred heat to forma dehydrogenation product.

Aspect 2: The method of aspect 1, wherein design of FBR apparatus with internal regenerator as riser.

Aspect 3: The method of aspect 1 or 2, wherein the method further comprises transferring a deactivated catalyst from the integrated fluidized bed reactor to the in-situ regenerator

Aspect 4: The method of any one of aspects 1-3, wherein method further comprises transferring a deactivated catalyst from the integrated fluidized bed reactor to the regenerator reactor.

Aspect 5: The method of any one of aspects 1-4, wherein method further comprises transferring the activated catalyst from the regenerator reactor to the integrated fluidized bed reactor.

Aspect 6: The method of any one of aspects 1-5, wherein the paraffin comprises from three to six carbons.

Aspect 7: The method of any one of aspects 1-6, wherein the paraffin comprises propane or isobutane, or a combination thereof.

Aspect 8: The method of any one of aspects 1-7, wherein the dehydrogenation product comprises propene, n-butene, n-pentene, iso-pentene, n-hexene, or iso-hexene, or a combination thereof.

Aspect 9: The method of any one of aspects 1-8, wherein the paraffin is dehydrogenated at a temperature ranging from 490° C. to 640° C.

Aspect 10: The method of any one of aspects 1-9, wherein the paraffin is dehydrogenated at a pressure ranging from 0.1 atmospheres to 3 atmospheres (0.01 MPa to 0.3 MPa).

Aspect 11: The method of any one of aspects 1-10, wherein the catalyst comprises a Cr-based catalyst or a Pt-based catalyst, or a combination thereof.

Aspect 12: The method of any one of aspects 1-11, wherein the catalyst comprises a promoter and is dispersed on aluminum oxide, silicon oxide, or zeolite, or a combination thereof.

Aspect 13: The method of any one of aspects 1-12, wherein the paraffin is dehydrogenated at a GHSV ranging from 100 h⁻¹ to 10000 h⁻¹ for a Cr-based catalyst and 100 h⁻¹ to 10000 h⁻¹ for a Pt-based catalyst.

Aspect 14: The method of any one of aspects 1-13, wherein the dehydrogenation of the paraffin in the integrated fluidized bed reactor to form a dehydrogenation product and activation of the deactivated catalyst in the regenerator reactor occurs simultaneously.

Aspect 15: The method of any one of aspects 1-14, wherein the catalyst is regenerated by burning coke on the catalyst surface at a temperature greater than the average temperature of the reactor in a stream comprising air, oxygen, and fuel gas.

Aspect 16: The method of any one of aspects 1-15, wherein the fluidized bed reactor is substantially isothermal.

Aspect 17: The method of any one of aspects 1-16, wherein the first longitudinal axis is parallel to the second longitudinal axis.

Aspect 18: The method of any one of aspects 1-17, wherein the first longitudinal axis and the second longitudinal axis are axially aligned, thereby forming a common longitudinal axis.

Aspect 19: An apparatus for dehydrogenation of a paraffin comprising a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space.

Aspect 20: The apparatus of aspect 19, wherein the dehydrogenation reactor further comprises a striping zone, wherein the striping zone is in fluid communication with the integrated fluidized bed reactor, and wherein the striping zone is connected to the regenerator reactor via a first connector.

Aspect 21: The apparatus of aspects 19 or 20, wherein the integrated fluidized bed reactor or the striping zone or a combination thereof comprises an integrated internal baffle or a perforated tray or a combination thereof.

Aspect 22: The apparatus of aspect 21, wherein the first connector comprises a gas injection system.

Aspect 23: The apparatus of any one of aspects 19-22, wherein the dehydrogenation reactor further comprises a catalyst collection zone, wherein the regenerator reactor has an outlet that is positioned in the catalyst collection zone, and wherein the catalyst collection zone is connected to the integrated fluidized bed reactor via a second connector.

Aspect 24: The apparatus of any one of aspects 19-23, wherein the first longitudinal axis is parallel to the second longitudinal axis.

Aspect 25: The apparatus of any one of aspects 19-24, wherein the integrated fluidized bed reactor comprises a feed distributor or a cleaning ring or a combination thereof.

Aspect 26: The apparatus of aspect 25, wherein the feed distributor or the cleaning ring or a combination thereof are connected one or more paraffin inlets.

Aspect 27: The apparatus of any one of aspects 19-26, wherein the integrated fluidized bed reactor is configured to contain an endothermic reaction.

Aspect 28: The apparatus of any one of aspects 19-27, wherein the regenerator reactor is configured to contain an exothermic reaction.

Aspect 29: The apparatus of any one of aspects 19-28, wherein the apparatus does not comprise an external regenerator reactor.

Aspect 30: The apparatus of any one of aspects 19-29, wherein the first longitudinal axis and the second longitudinal axis are axially aligned, thereby forming a common longitudinal axis.

Aspect 31: The method of any one of aspects 1-18 or apparatus of any of aspects 19-30, wherein the first longitudinal axis and the second longitudinal axis are axially aligned, thereby forming a common longitudinal axis.

Aspect 32: The apparatus of any one of aspects 19-30, wherein the apparatus equipped with secondary distributor at upper part in entrainment section of regenerated catalyst.

Aspect 33: The apparatus of any one of aspects 19-30 and 32, wherein the apparatus equipped with secondary distributor for feed gas.

1. A method for dehydrogenation of a paraffin comprising:
providing a dehydrogenation reactor comprising an integrated fluidized bed reactor and in-situ regenerator, wherein the integrated fluidized bed reactor has a first longitudinal axis and comprises an inner surface defining an interior space, wherein the in-situ regenerator has a second longitudinal axis and is positioned at least partially within the interior space;
activating the deactivated catalyst by regeneration with any oxygen containing gas/fluid performing an exothermic reaction and transferring the heat to the integrated fluidized bed reactor for reaction; and dehydrogenation of paraffin present in the integrated fluidized bed reactor by performing an endothermic reaction, and at least a portion of the transferred heat to form a dehydrogenation products.

2. (canceled)

3. The method of claim 1, further comprising transferring a deactivated catalyst from the integrated fluidized bed reactor to the in-situ regenerator.

4. The method of claim 1, further comprising transferring the activated catalyst from the regenerator reactor to the integrated fluidized bed reactor.

5. The method of claim 1, wherein the paraffin comprises from three to six carbons wherein the dehydrogenation product comprises propene, n-butene, n-pentene, iso-pentene, n-hexene, or iso-hexene, or a combination thereof, and wherein the catalyst comprises a Cr-based catalyst or a Pt-based catalyst, or a combination thereof.

6. (canceled)

7. (canceled)

8. The method of claim 1, wherein the paraffin is dehydrogenated at a temperature ranging from 490° C. to 640° C. and wherein the paraffin is dehydrogenated at a pressure ranging from 0.1 atmospheres to 3 atmospheres (0.01 MPa to 0.3 MPa).

9. (canceled)

10. (canceled)

11. (canceled)

12. (canceled)

13. The method of claim 1, wherein the dehydrogenation of the paraffin in the integrated fluidized bed reactor to form a dehydrogenation product and the activation of the deactivated catalyst in the regenerator reactor occurs simultaneously.

14. The method of claim 1, wherein the catalyst is regenerated by burning coke on the catalyst surface at a temperature greater than the average temperature of the reactor in a stream comprising air, oxygen, and fuel gas.

15. The method of claim 1, wherein the fluidized bed reactor is substantially isothermal.

16. (canceled)

17. (canceled)

18. An apparatus for dehydrogenation of a paraffin comprising a dehydrogenation reactor comprising an integrated fluidized bed reactor and a regenerator reactor, wherein the integrated fluidized bed reactor has a first longitudinal axis and comprises an inner surface defining an interior space, wherein the regenerator reactor has a second longitudinal axis and is positioned at least partially within the interior space.

19. The apparatus of claim 18, wherein the dehydrogenation reactor further comprises a striping zone, wherein the striping zone is in fluid communication with the integrated fluidized bed reactor, and wherein the striping zone is connected to the regenerator reactor via a first connector.

20. The apparatus of claim 18, wherein the integrated fluidized bed reactor or the striping zone or a combination thereof comprises an integrated internal baffle or a perforated tray or a combination thereof.

21. The apparatus of claim 20, wherein the first connector comprises a gas injection system.

22. The apparatus of claim 18, wherein the dehydrogenation reactor further comprises a catalyst collection zone, wherein the regenerator reactor has an outlet that is positioned in the catalyst collection zone, and wherein the catalyst collection zone is connected to the integrated fluidized bed reactor via a second connector.

23. The apparatus of claim 18, wherein the first longitudinal axis is parallel to the second longitudinal axis.

24. The apparatus of claim 18, wherein the first longitudinal axis and the second longitudinal axis are axially aligned, thereby forming a common longitudinal axis.

25. The apparatus of claim 18, wherein the integrated fluidized bed reactor comprises a feed distributor or a cleaning ring or a combination thereof, wherein the feed distributor or the cleaning ring or a combination thereof are connected one or more paraffin inlets.

26. (canceled)

27. The apparatus of claim 18, wherein the integrated fluidized bed reactor is configured to contain an endothermic reaction.

28. The apparatus of claim 18, wherein the regenerator is configured to contain an exothermic reaction or wherein the apparatus does not comprise an external regenerator reactor.

29. (canceled)

30. The apparatus of claim 18, wherein the apparatus is equipped with a secondary distributor at an upper part in an entrainment section of regenerated catalyst or wherein the apparatus equipped with secondary distributor for feed gas.

31. (canceled)

32. The method of claim 15, further comprising controlled the reactor temperature with an outside thermal reservoir, wherein the outside thermal reservoir is a heat exchanger or a regeneration riser.
